(19) United States
(12) Patent Application Publication
(10) Pub. No.: US 2026/0306086 A1
(43) Pub. Date: Oct. 8, 2026

(54) SURGICAL SPATULA

(71) Applicant: MINDER S.r.l., Brescia (BS) (IT)

(72) Inventors: Elena ROCA, Rezzato (BS) (IT); Giorgio RAMORINO, Brescia (BS) (IT); Gianluigi DALÈ, Brescia (BS) (IT)

(73) Assignee: MINDER S.r.l., Brescia (BS) (IT)

(21) Appl. No.: 19/478,857

(22) PCT Filed: Jun. 6, 2024
(86) PCT No.: PCT/IB2024/055521

§ 371 Date: Oct. 27, 2025

(30) Foreign Application Priority Data
Jun. 16, 2023 (IT) 102023000012432

(51) Int. Cl. A61B 17/02; A61B 17/00; A61B 90/00; A61B 90/30

(52) U.S. Cl. CPC A61B 17/02; A61B 90/30; A61B 2017/00862; A61B 2017/00946; A61B 2090/0807; A61B 2090/0817

(57) ABSTRACT

A manual surgical spatula is made of a single-body with substantially laminar shape. When not in use, the spatula is arched whereas, during use, to move human tissues, the spatula is susceptible to elastic bending in which it is straightened it into a flat configuration, in response to the strength the patient's tissues exerting at an end of the spatula. The spatula is equipped with one or more tactile and/or visual indicators aligned along the longitudinal axis, each indicating a point of grip uniquely corresponding to a value of the bending strength of the spatula. The spatula when held at a specific tactile and/or visual indicator, the flattening of the spatula occurs in response to a pressure value uniquely corresponding to that specific tactile and/or visual indicator. Accordingly, a surgeon has objective and quantitative feedback of the pressure exerted on the tissues.

### CROSS-REFERENCE TO RELATED APPLICATIONS

This application is a § 371 U.S. National Phase of PCT/IB2024/055521, filed Jun. 6, 2024, which claims priority to Italian Patent Application No. 102023000012432, filed Jun. 16, 2023, the entire contents of all of which are incorporated by reference herein as if fully set forth.

### FIELD OF THE INVENTION

The present invention relates to a surgical spatula and, in particular, a surgical spatula for vascularized tissues.

### STATE OF THE ART

In the surgical field, the use of traction instruments, also called dilators or retractors, is known, which have the function to move the tissues of the patient undergoing surgery, at the surgical site, to give the surgeon access, e.g., to a tumor to be removed or tissues to be examined.

Among the most widely used traction instruments are spatulas which, like all surgical instruments, are subjected to certification and marketing authorization obligations.

A traditional manual spatula is made like a medical steel foil, i.e. as an elongated element of negligible thickness compared to length and width. The spatulas are flat, with a rectangular cross-section, or corrugated or spoon-shaped, with a U-shaped cross-section.

The manual spatula is held by the surgeon, with one hand, to be inserted into the surgical site and to push the tissues that need to be momentarily moved to access the underlying area of interest.

Manual spatulas are used for a variety of surgical procedures but are particularly relevant in craniotomies, in which portions of the brain need to be temporarily moved to access the area of interest, due to the delicacy of the brain tissue. Indeed, applying excessive pressure to the brain tissue, which is defined as brain retraction pressure BRP, can easily cause harm to the patient. In particular, in cases of neurosurgery of the cranial base, tumor lesions may be located in very deep and dangerous brain areas to reach; in these cases, it is therefore necessary to create an adequate surgical space by using one or more surgical spatulas, but this often results in the risk of damaging noble brain structures as well as the parenchyma.

A further drawback is that the edges of traditional metal manual spatulas have proven to be particularly dangerous, in particular the pressures exerted by the spatula on the parenchyma are always greater on the edges and, during their movement, the surgeon often makes a displacement that also involves a rotation of the spatula with a further localized increase in BRP in the edges.

Possible brain retraction damages may include contusions, hematomas, hemorrhages and nerve lesions that set up patterns of direct iatrogenic damage, or parenchymal ischemia episodes due to metabolic hemodynamic changes. All of these complications can consequently affect the good outcome of the surgery and thus nullify the good outcome of the patients' surgical procedure. In the literature, incidence data of complications from surgical retraction are set forth, which are as high as 45% if post-operative radiological investigations as damage detectors are considered. (Recinos P F, Raza S M, Jallo G I, Recinos V R. Use of a minimally invasive tubular retraction system for deep-seated tumors in pediatric patients: Technical note. J. Neurosurg Pediatr. 2011;7(5):516-521. Doi:10.3171/2011.2.PEDS 10515. Serarslan Y, Cokluk C, Aydin K, Iyigun O. Soft micro-balloon paddy for brain retraction in the protection of neuronal tissue. Minim Invasive Neurosurg MIN. 2006;49(6):373-375. doi:10.1055/s-2006-955067. Singh L, Agrawal N. Stitch retractor—simple and easy technique to retract brain. World Neurosurg. 2010;73(2):123-127. doi:10.1016/j.surneu.2009.01.031).

The threshold value of the BRP pressure exerted on the tissues, beyond which tissues are damaged, is not universally recognized but depends on the nature of the tissues, the circumstances and the duration of application, i.e. the duration of retraction.

For example, in the papers:
Rosenørn J, Diemer N H.—Reduction of regional cerebral blood flow during brain retraction pressure in the rat. Journal Neurosurg. 1982; 56(6): 826-829. doi:10.3171/jns.1982.56.6.0826;
Rosenørn J, Diemer N H. The influence of intermittent versus continuous brain retractor pressure on regional cerebral blood flow and neuropathology in the rat. Acta Neurochir (Wien). 1988;93(1-2):13-17. doi: 10.1007/BF01409896, the authors describe that keeping a BRP pressure in the range of 30-40 mmHg for 15 minutes causes a significant reduction in the regional Cerebral Blood Flow rCBF resulting in brain damages, whereas performing the same experiments with the intermittent application of BRP pressure=40 mmHg for 5-7 consecutive minutes, with an interval of 1 minute, no damage is detected.

In general, tests have inferred that intermittent application of BRP pressure, no longer than 7 minutes with maximum values of about 40 mmHg, is preferable for humans to ensure that no damages to brain tissues are caused. Should it be necessary to apply PRC pressure for longer time intervals, e.g. 10 consecutive minutes, before an interval, then the PRC pressure value must necessarily be lower, e.g. 30 mmHg.

However, these are not universally valid intervals and, in practice, the surgeon is entrusted with the task of assessing the intensity and duration of the intermittent BRP pressure, based on the needs of the surgical procedure performed at that time.

It has also been noticed that some lesions are immediately visible during surgery when the retraction damage has already occurred. However, intraoperative detection of any damages allows the surgeon to adjust the push applied. A different matter is the case for lesions that are not visible to the surgeon either macroscopically or microscopically: these are specifically those parenchyma damages that are not immediately detectable intraoperatively but can only be identified retrospectively, i.e. after the operation has been completed, with post-operative CT or MRI radiological investigations. The aforesaid damages are therefore more dangerous because the surgeon cannot detect them during the procedure and, therefore, cannot adapt the pressure exerted on the brain.

The criticalities just above may be even more evident in children, as the space available in a child's skull is more limited than that available in adults and, therefore, more pressure must be exerted on the brain tissues to move them, and the likelihood of error is greater.

U.S. Pat. No. 6,093,145 describes a surgical spatula for brain procedures that comprises an inner metal core with rectangular cross-section, coated with a soft, elastically deformable material equipped with flexible wings and rounded edges. The coating material is preferably silicone. This solution allows to achieve a spatula whose central portion has hardness between 50 and 70 Shore and in which the edges have hardness between 20 and 40 Shore, therefore far less dangerous than the edge of a traditional uncoated spatula.

JP 2005 323793A describes a spatula telescopically constrained to a cylindrical grip that also functions as a container of surgical fluids to be released during the procedure and as a sheath for guiding surgical instruments. This solution minimizes the length of the edges of the spatula that, at any given time, come into contact with the patient's tissues, as the edges in contact with the tissues are only those of the portion of spatula that is extracted from the grip.

The effectiveness of manual spatulas, as those just described, substantially depends on the surgeon's sensitivity and accuracy, i.e. an error made by the surgeon when using a manual spatula, exceeding the threshold value of the BRP pressure during the retraction (spatulation) of the tissues, cannot be compensated or mitigated in any way: traditional manual surgical spatulas substantially transmit to the tissues the same pressure that the surgeon exerts on the spatula.

In order to provide feedback to the surgeon, sensor-equipped spatulas have recently been proposed, i.e. provided with at least one pressure sensor and a corresponding electronic control circuit PCB; the sensor is positioned at the end of the spatula intended to be inserted between the tissues and generates an electrical signal indicating the BRP pressure exerted on the sensor (by the patient's tissues). The control circuit acquires and processes the signal generated by the pressure sensor and emits an acoustic and/or light signal when a threshold value is exceeded. This way, the surgeon has feedback of the PRC pressure applied to the spatula and can adapt it to the current conditions in order not to exceed the threshold value.

Spatulas equipped with pressure sensor are described, e.g., in JP 6856198, CN 204181665(U), CN 115227300A and CN 114129206A.

Therefore, if on the one hand manual surgical spatulas are simple to make at low costs but do not provide feedback to the surgeon and do not leave any room for human error, on the other hand, sensor-equipped surgical spatulas provide feedback but are more complex and, therefore, difficult to make and expensive and have generally larger encumbrances than a traditional surgical spatula, due to the fact that they house the sensor, the control circuit and related connections.

US 2007/208226 describes a single-body, manual surgical spatula that extends along a longitudinal axis between a first end, at which the grip is provided, and a second operative end. The thickness of the surgical spatula is negligible compared to the length and is substantially equal to the width of the surgical spatula, as a part of the surgical spatula has a substantially square cross-section. The spatula is made of polycarbonate and can be functionally coupled to a LED light source at the grip. The light travels through the polycarbonate, from the LED light source toward the opposite end of the surgical spatula. At a flattened portion of the surgical spatula, which is proximal to the operative end, there is a facetted surface machined like a Fresnel lens, having the function of directing the light from inside the surgical spatula outwards and in front of the operative end, as a headlight would do. The facetted portion then allows to illuminate the tissues during the use of the spatula, conveying the light generated by the LED light source.

GB 1242374 describes a spatula whose operative end is substantially rigid and has a plurality of holes. The holes can be harmful to brain parenchyma, as they can cause micro-traumas to the tissue, which is not solid but soft, and can then creep into the holes, the tissue therefore suffering damages.

U.S. Pat. No. 3,888,117 describes a rigid surgical spatula, with a steel core, equipped with a pressure sensor. In turn, the sensor comprises a number of beads inserted into appropriate holes arranged along the spatula, with constant pitch. The beads are enclosed in an insulating casing and, by moving within the respective hole, can come into contact with a conductive tape inside the spatula, thus closing an electrical circuit and allowing a signal to be generated. The beads are electrically connected to a single longitudinal wire and, for this reason, are not selectively retractable into their respective holes.

US 2018/317902 describes a flexible surgical spatula equipped with a plurality of light sources powered by a battery, such as LEDs, arranged along the surgical spatula at a constant pitch, the spatula being equipped with suction tubes located on the sides of the surgical spatula, which can be connected to an external suction source. The light sources have the function to illuminate the surgical site and to provide the surgeon with an indication of the depth to which the surgical spatula has been pushed between the patient's tissues.

### SUMMARY

The technical problem underlying the present invention is therefore to provide a manual surgical spatula, i.e. devoid of electronic sensors and circuits, that is simple in structure, reliable, ergonomic, inexpensive and that allows to overcome the limitations of traditional solutions, by limiting or preventing altogether the exceeding of the threshold pressure beyond which the patient's vascularized tissues are damaged, during the retraction (spatulation), i.e. a spatula that provides adequate feedback to the surgeon.

It is a further object of the present invention to provide a surgical spatula for brain procedures, i.e. craniotomies in adults and children, which does not prevent the surgeon from seeing the spatulated tissues.

Thus, the present invention concerns a surgical spatula as described below.

The surgical spatula is of manual type, i.e. devoid of any electronic sensors and respective control circuit, and is made as a single body, i.e. as a single element and not an assembly of assembled components.

The spatula extends along a longitudinal axis, between a first end and a second end, and has a substantially laminar or flattened shape, meaning that the thickness of the spatula is smaller than its length and width, preferably negligible. The second end is the operative end, the one intended to come into contact with the patient's tissue during the surgical procedure. Preferably, the thickness of the spatula is negligible compared to the length and width.

The spatula can be held by the surgeon with one hand between the first end and the second end.

Under normal conditions, i.e. at rest, when not in use, the spatula is arched or, in other words, is concave; during use, however, when the surgeon uses the spatula to move human tissues, the spatula is susceptible to elastic bending that tend to flatten it, i.e. straighten it, by bringing it into a flat configuration in response to the pressure (strength) that the patient's tissues exert at an end of the spatula.

Thus, the spatula is flexible from a concave or arched configuration to a flat configuration corresponding to reaching the pressure threshold value in response to stresses imparted by the surgeon during spatulation. Should it be desired to go further with the bending, reversing the curvature of the spatula from the flat configuration onwards, one is aware of being over the threshold value.

The spatula is equipped with one or more tactile and/or visual indicators aligned along the longitudinal axis, each indicating a point of grip uniquely corresponding to a precise value of the bending strength of the spatula, which is quantitatively known a priori and specified by the manufacturer, i.e. a pre-set value measured and measurable, which is the same for all spatulas manufactured.

The bending strength value of the spatula, which corresponds to each of the tactile and/or visual indicators, is provided along with the illustrative material on the operation of the spatula, e.g. along with an instruction manual or a certificate of conformity/approval, but it can also be written directly on the surgical spatula.

Advantageously, the surgical spatula can be certified by the manufacturer to offer an exact and unique match between each tactile and/or visual indicator and the corresponding bending strength value. This allows the surgeon to constantly keep the control of the maximum pressure exerted on the tissues during the surgical procedure.

In simple terms, tactile and/or visual indicators constitute a graded scale of the bending strength of the surgical spatula, i.e. the strength that the surgical spatula offers to its straightening; the straightening involves having reached the maximum pressure value corresponding to having gripped the spatula at that precise tactile/visual indicator.

This arrangement allows to make the spatula in such a way that by holding it at a specific tactile and/or visual indicator, the flattening of the spatula occurs when reaching a pressure value uniquely corresponding to that specific tactile and/or visual indicator. This means that the surgeon, by squeezing the spatula at a tactile and/or visual indicator, is applying a pressure to the patient's tissues with the second end of the spatula and, in particular, with a pre-set contact area of the second end; the pressure is less than or equal to a corresponding threshold value at which the complete straightening (flattening) of the spatula is achieved.

The pre-set contact area is a working area that is used to apply pressure on the tissues, as an alternative to the edges of the spatula. In practice, an area other than the contact area is not used for several main reasons. First of all, the use of the edges of the spatula would easily cause lesions to the brain parenchyma and may induce unwanted twists of the spatula. Additionally, the brain has a substantially curved shape, therefore, during a surgical procedure, when the brain has to be spatulated to reach the depth, the entire contact area of the spatula is rested to achieve maximum anatomical compatibility with the brain and maximum ergonomics for the surgeon.

The tactile and/or visual indicators therefore allow to calibrate the pressure exerted on the patient's tissues, according to quantitative and non-qualitative values, with clear benefit for the good outcome of the surgical procedure.

The spatula therefore offers passive, but immediate, feedback to the surgeon. Unlike a sensor-equipped solution, which provides an audible or visual alarm signal, the spatula according the present invention provides the surgeon with purely mechanical/visual feedback: when the surgeon exerts on the patient's tissues, with the contact area of the second end, a pressure sufficient to straighten the spatula, i.e. to flatten it, then he is aware that he has reached the pressure threshold value corresponding to the specific tactile and/or visual indicator at which he is holding the spatula. Beyond the specific bending strength value reached by straightening the spatula, the surgeon is aware that the pressure being exerted on the patient's brain tissues is greater than the value corresponding to the specific tactile/visual indicator the surgeon is holding at that time, and he is therefore also aware that increasing the pressure further could may damage the patient's brain tissues. In other words, the surgeon constantly keeps the control of the pressure exerted.

Clearly, it is possible to make the spatula with a single tactile and/or visual indicator, but preferably the spatula is made with multiple indicators.

In the case of a single tactile and/or visual indicator, the user, i.e. the surgeon, has three gripping positions available: one at the indicator, one immediately forward the indicator and one immediately behind the indicator. This configuration therefore allows to have three threshold values of (maximum) pressure applied to the tissues when the spatula is straightened.

Clearly, by squeezing the spatula at an intermediate portion between two consecutive tactile and/or visual indicators, the feedback provided to the surgeon corresponds to a pressure value intermediate between the two threshold values uniquely corresponding to the two consecutive tactile and/or visual indicators.

Preferably, the tactile and/or visual indicators are four and correspond to values of maximum pressure (exerted on the patient's tissues), at one end of the spatula, equal to 15 mmHg, 22 mmHg, 30 mmHg and 45 mmHg, equivalent to 1999.83 Pa, 2933.084 Pa, 3999.66 Pa and 5999.49 Pa, respectively.

The surgical spatula according to the preferred embodiment is normally arched, i.e. when not in use it extends along an arc of circumference defined by a radius R, e.g. equal to 150-200 mm, and an angle at the center, e.g. equal to 50°-60°. Considering this geometry, the angular pitch between the tactile and/or visual indicators, which is calculated with the spatula arched at rest, and with reference to the angle at the center, corresponds to 4°-6°. Thus, with this angular pitch, the distance between the tactile and/or visual indicators is equal to about 5 mm.

Preferably, a first tactile and/or visual indicator intercepts an angle at the center equal to 22° with the first end of the spatula and the fourth tactile and/or visual indicator intercepts an angle at the center equal to 36° with the first end of the spatula.

In the preferred embodiment, the tactile and/or visual indicators are bosses protruding from the top surface of the spatula. The bosses can be 0.5 to 1 mm thick so that they can be felt when touched by the surgeon with gloves on.

Preferably, the surgical spatula is 15-20 cm long, 1-2 cm wide and 1-4 mm thick. The curvature radius of the resting spatula is in the range 150-200 mm.

Preferably, the surgical spatula is made of a material having stiffness between 2 and 4 GPa.

More preferably, the spatula is made of a transparent polymer material, so that the surgeon may observe the spatulated tissues directly and at the same time as the surgical action and verify he is not applying excessive pressure. In particular, it should be noted that conventional traditional spatulas, made of non-transparent metal, exert a retraction that does not show the underlying retracted parenchyma at that time, but it is only after the removal of the spatula that any damages can be seen on the brain.

In the preferred embodiment, the spatula is made of a material selected from polycarbonate (PC), polymethyl methacrylate (PMMA), polystyrene (PS), low density polyethylene (LDPE). These are transparent polymers.

In an embodiment, the surgical spatula comprises a section narrowing whose function is to allow the relative twisting of the two portions of the spatula, which are on opposite sides of the section narrowing, to each other. The section narrowing then defines an elastic joint that limits or prevents the transfer of a twisting force from the portion of spatula held by the surgeon to the portion of spatula in contact with the patient's tissues, and this allows to limit the pressure exerted on the tissues by the edges of the spatula.

In the circumstance that the surgeon inadvertently tilts the gripped portion of spatula with respect to the tissue to be spatulated, the part of the spatula in contact with the patient's tissues would indeed rotate less than the part gripped by the surgeon and, therefore, the spatula would not be working exclusively or mainly on the edge.

The section narrowing therefore works as a twist limiter in order to keep the portion of spatula, in contact with the tissues, resting as flat as possible with the tissues, or to limit or prevent the spatula from cutting with one of its edges.

Preferably, the section narrowing is formed by making the spatula with two notches, or indentations, opposite the longitudinal axis, each cut into an edge of the spatula. The section narrowing is the portion of spatula that is included between the two notches. More preferably, the two notches extend orthogonally to the longitudinal axis of the spatula, toward each other, at a portion of spatula that is intermediate between two tactile and/or visual indicators, e.g. between the third and fourth indicators.

In other words, the portion of spatula intermediate between the two notches defines an elastic hinge that allows the relative rotation of the portion of spatula which is on one side of the notches, held by the surgeon, with respect to the portion of spatula opposite the notches, which is intended to come into contact with the patient's tissues.

Preferably, the section narrowing extends along the longitudinal axis by 2-5 mm and is between 40% and 50% of the length of the spatula, which is considered from the second end (i.e. the end opposite that of the grip), i.e. closer to the second end than the first end.

Another optional characteristic of the spatula is the following: by making the spatula of a transparent material, such as polycarbonate, the spatula can be used to direct light into the surgical site. It is sufficient to equip the spatula with a light source at the first end, directed in such a way that the rays of light propagate inside the spatula, bouncing off its inner surfaces until reaching the second end, from which they then come out to illuminate the tissues. In other words, the spatula behaves like an optical fiber and the light produced by the light source is reflected where it is most needed, i.e. on the tissues at the contact area with the spatula, to the benefit of the surgeon.

### BRIEF DESCRIPTION OF THE FIGURES

Further characteristics and advantages of the invention will be more evident from the review of the following specification of some preferred, but not exclusive, embodiments depicted for illustration purposes only and without limitation, with the aid of the attached drawings, wherein:

FIG. 1 is a perspective view of a surgical spatula according to the present invention;

FIG. 2 is a top plan view of the surgical spatula shown in FIG. 1;

FIG. 3 is a side view of the surgical spatula shown in FIG. 1;

FIG. 4 is a photograph showing a perspective view of a surgical spatula according to the present invention, during the retraction of swine's brain tissue, in a first configuration corresponding to 15 mmHg of applied pressure, which is equal to about 1999 Pa;

FIG. 5 is a photograph showing a perspective view of a surgical spatula according to the present invention during the retraction of swine's brain tissue, in a second configuration corresponding to 22 mmHg of applied pressure, which is equal to about 2933 Pa;

FIG. 6 is a photograph showing a perspective view of a surgical spatula according to the present invention during the retraction of swine's brain tissue, in a third configuration corresponding to 30 mmHg of applied pressure, which is equal to about 3999 Pa;

FIG. 7 is a photograph showing a perspective view of a surgical spatula according to the present invention during the retraction of swine's brain tissue, in a fourth configuration corresponding to 45 mmHg of applied pressure, which is equal to about 5999 Pa;

FIG. 8 is a collage of six photographs of swine's brain tissue magnified by a microscope (40× magnification), before the retraction and after the retraction with a spatula according to the known art;

FIG. 9 is a collage of twelve photographs of swine's brain tissue magnified by a microscope (40× magnification), before the retraction and after the retraction with a spatula according to the present invention; and

FIG. 10 is a schematic view of a longitudinal section of the spatula shown in FIG. 1, equipped with a light source.

### DETAILED DESCRIPTION OF THE PREFERRED EMBODIMENTS

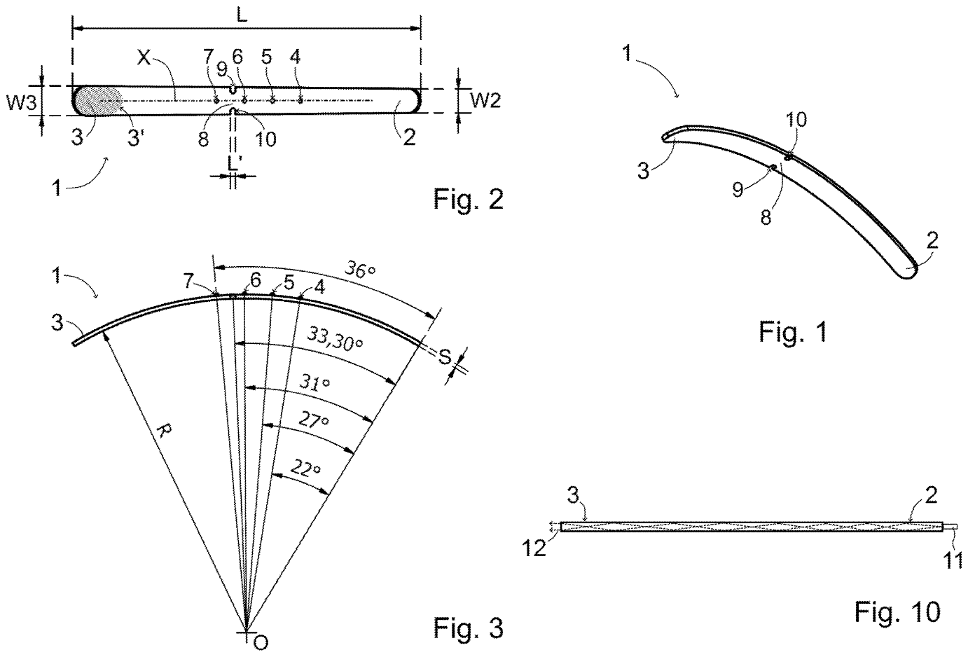

A surgical spatula 1 according to the present invention, henceforth for simplicity only spatula 1, is shown in perspective, from below, in FIG. 1. The spatula 1 has single-body, i.e. defined by a single element made of one piece and not as an assembly of several components. The spatula 1 has a laminar configuration, meaning that the length and width are much greater than the thickness. The spatula 1 extends from a first end 2 intended to be held by the surgeon or, in any case, intended to be gripped in one hand of the surgeon, and a second end 3 opposite the first end 2, which is intended to exert pressure on the patient's tissues to perform retraction of the tissues.

In particular, in FIG. 2 on the second end 3, an area 3′ corresponding to the ideal area of contact of the spatula 1 with the tissues to be retracted is shaded, which can also be defined as the pre-set contact area 3′ or working area 3′ of the end 3 of the spatula 1. In the example shown in the figures, the contact area 3′ has an extent equal to about 315 mm². As will be specified hereinafter, the values of the pressure applied during the retraction refer to the ideal condition of contact of the spatula 1 with the tissues on the entire contact area 3′.

FIG. 2 shows the spatula 1 in a top plan view with the spatula 1 straightened (flattened) and FIG. 3 is a side view of the spatula 1.

As can be seen in FIGS. 1 and 3, the spatula 1 is normally arched, meaning that when not in use it remains curved in an arc. As will be clear below, the spatula 1 is susceptible during use to elastic bending which may cause the complete straightening of the spatula 1, until reaching a level configuration, precisely flattened. The reference R denotes the radius of curvature, which for the spatula 1 in the example shown in the figures is equal to 167 mm, but in general can be between 150 mm and 200 mm.

In the example shown in FIGS. 1-3, the spatula 1 has a length L of about 17 cm; the width W2 of the first end 2 is equal to 12 mm and the width W3 of the second end 3 is equal to 15 mm, i.e. the second end 3 is slightly wider than the first end 2. In general, width dimensions can range from 1 cm to 2 cm. The thickness S of the spatula 1 shown in the figures is equal to 2 mm and in general can range from 1 mm to 4 mm.

The spatula 1 is transparent and preferably made by molding polycarbonate (PC). Alternatively, other transparent polymers, such as polymethyl methacrylate (PMMA), polystyrene (PS), low density polyethylene (LDPE) may be used.

Ideally, the spatula 1 is made of polycarbonate with stiffness between 2 and 4 GPa.

The spatula 1 is provided with one or more tactile and/or visual indicators 4-7. In the example shown in FIGS. 1-3, the spatula 1 comprises four tactile and/or visual indicators 4-7; these are bosses 4-7 protruding from the top surface of the spatula 1. The bosses 4-7 are aligned along the longitudinal axis X of the spatula and are 0.5 to 1 mm thick. The bosses 4-7 are visible to the surgeon and can also be detected by touch through the gloves. Each boss 4-7 has the task of indicating a precise point of grip of the spatula 1, uniquely corresponding to a precise value of the bending strength of the spatula 1, as will be explained below.

The bosses 4 are arranged along the longitudinal axis X according to pitch spacing. With reference to FIG. 3, O denotes the vertex of the angle at the center defined by the spatula 1 at rest, and R denotes the radius of curvature. The first boss 4 intercepts an angle at the center equal to 22° with the first end 2 of the spatula 1, the second boss 5 intercepts an angle at the center equal to 27° with the first end 2 of the spatula 1, the third boss 6 intercepts an angle at the center equal to 31° with the first end 2 of the spatula 1 and the fourth boss 7 intercepts an angle at the center equal to 36° with the first end 2 of the spatula 1.

Thus, the first boss 4 and the second boss 5 are spaced by an angular pitch equal to 5°, the second boss 5 and the third boss 6 are spaced by an angular pitch equal to 4° and the third boss 6 and the fourth boss 7 are spaced by an angular pitch equal to 5°.

The spatula 1 also comprises a section narrowing 8 which, in the example shown in FIGS. 1-3, is located between the third boss 6 and the fourth boss 7. The section narrowing 8 is achieved by providing two opposite notches 9 and 10 on the edges of the spatula 1. The notches 9 and 10 are 1-2 mm deep, being directed toward each other, and have a longitudinal extent L′ equal to 2-5 mm. The section narrowing 8 is at a distance, calculated from the second end 3, between 40% and 50% of the length L of the spatula 1.

Each tactile and/or visual indicator 4-7 corresponds to a point of grip at which the surgeon holds the spatula 1, at which the spatula 1 provides a given strength to bending, which is quantitatively known a priori and certified by the manufacturer. In other words, the tactile and/or visual indicators 4-7 constitute a graded scale of the bending strength of the spatula, i.e. the strength that the spatula 1 provides when it is straightened and, therefore, a graded scale of the pressure exerted at all times on the tissues retracted with the same spatula 1.

For example, the correctness of the certification can be detected by performing a test with a pressure sensor; the contact area of the spatula 1 is rested on the pressure sensor and a pressure is applied until achieving the complete straightening and flattening of the spatula. At this point, the pressure exerted on the sensor is measured, which must correspond to the certified pressure.

When the surgeon performs the retraction of the tissues with the spatula 1, the spatula tends to straighten, bending from the arched configuration shown in FIG. 3 to a flat configuration as that shown in FIG. 2, with no chance of further bending, switching from concave to convex. Each tactile and/or visual indicator 4-7 therefore provides an indication of the maximum pressure exerted on the tissues during the retraction, when the spatula 1 is straightened.

For the example shown in the figures, the four tactile and/or visual indicators 4-7 correspond to maximum values of pressure equal to 15 mmHg, 22 mmHg, 30 mmHg and 45 mmHg, respectively. These values correspond to the pressure exerted in the circumstance where the surgeon rests the entire contact area 3′ on the tissues and not only a part of the end 3′ or only the edges.

The section narrowing 8 constitutes an elastic hinge which helps to allow the relative rotation of the portion of spatula held by the surgeon, with respect to the portion of spatula 1 in contact with the retracting tissues. In other words, the section narrowing 8 is a twist limiter that limits the transmission of twists to the portion of spatula 1 in contact with the tissues, in order to prevent the spatula 1 from cutting with one of its edges.

The function of the bosses 4-7 and the section narrowing 8, and in general the operation of the spatula 1, will be explained with reference to FIGS. 4-7.

FIGS. 4-7 are photographs related to a craniotomy experiment on an anesthetized swine. In particular, the photographs 4-7 show the swine's brain after opening the dura mater, the thick outermost tissue that protects the spinal cord and contains the cerebrospinal fluid. Therefore, retraction tests of the parenchyma P were carried out with a spatula 1′ according to the present invention, which is equipped with four visual indicators 4-7 colored in black on the transparent plastic material of the spatula 1′.

FIG. 4 shows a moment during the retraction of the parenchyma P: the surgeon grips the spatula 1′ in the left hand, between the thumb and forefinger, in such a way that the first visual indicator 4 remains barely visible. In this condition, the surgeon is aware that, by keeping the contact on the entire contact area 3′, the pressure applied to the parenchyma is certainly less than 15 mmHg, since the spatula 1 is not straight but still retains some curvature. The surgeon is also aware that by increasing the push on the spatula 1, it will reach the pressure of 15 mmHg by the time the spatula 1 is fully straightened. Thus, the surgeon who wished to apply a pressure of constantly less than 15 mmHg to the parenchyma P, should only have to carry out the retraction of the tissues without fully straightening the spatula 1′.

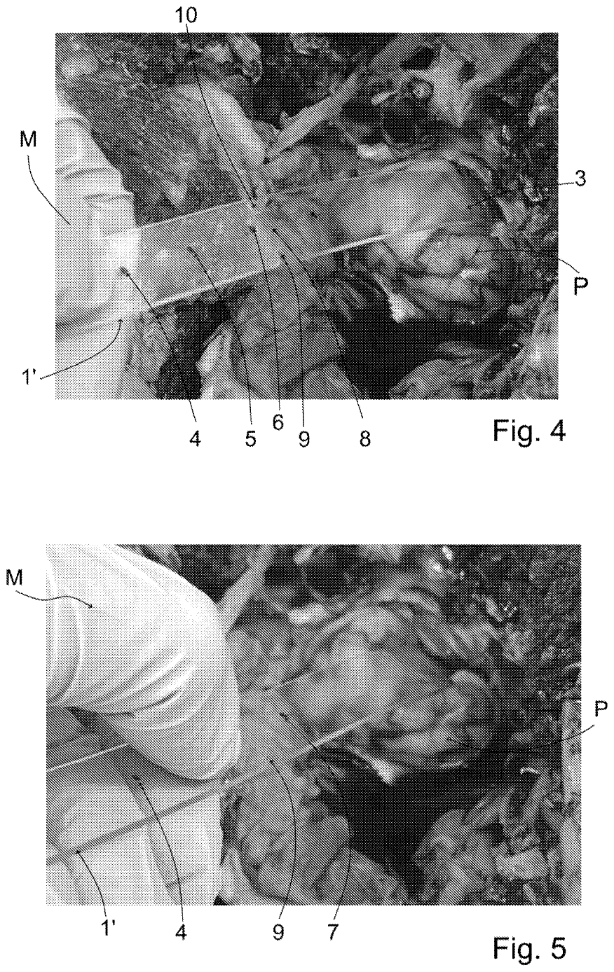

FIG. 5 shows another moment during the retraction of the parenchyma P: the surgeon grips the spatula 1′ in the left hand, between the thumb and forefinger, at the second visual indicator 5. In this condition, the surgeon is aware that, by keeping the contact on the entire contact area 3′, the pressure applied to the parenchyma will be at most equal to 22 mmHg when the spatula 1 is fully straightened. Thus, the surgeon who wished to apply a pressure of constantly less than 22 mmHg to the parenchyma P, should only have to carry out the retraction of the tissues without fully straightening the spatula 1′.

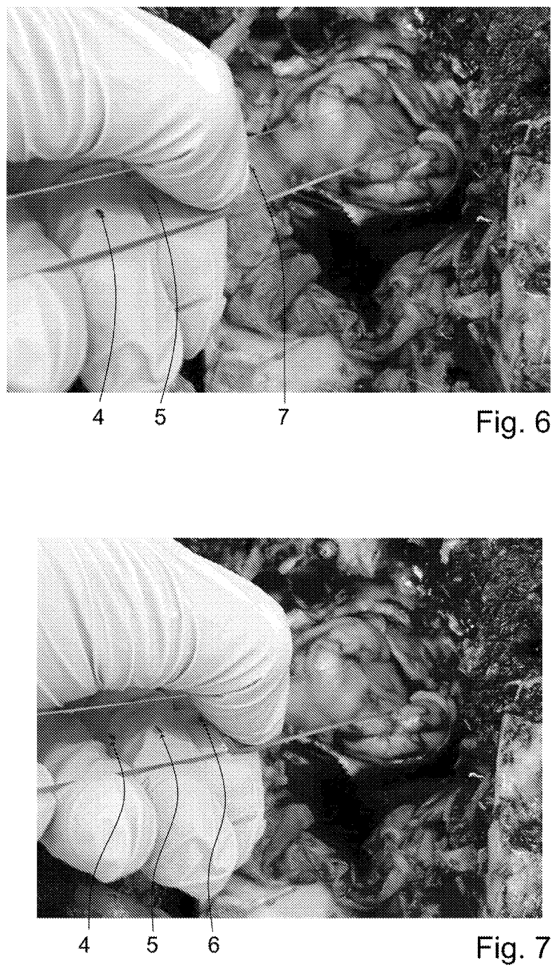

FIG. 6 shows another moment during the retraction of the parenchyma P: the surgeon grips the spatula 1′ in the left hand, between the thumb and forefinger, at the third visual indicator 6. In this condition, the spatula 1′ is fully straightened and the surgeon is aware that, by keeping the contact on the entire contact area 3′, the pressure applied to the parenchyma is exactly equal to 30 mmHg. Thus, the surgeon who wished to apply a pressure of constantly less than 30 mmHg to the parenchyma P, should only have to carry out the retraction of the tissues without fully straightening the spatula 1′.

FIG. 7 shows another moment during the retraction of the parenchyma P: the surgeon grips the spatula 1′ in the left hand, between the thumb and forefinger, at the fourth visual indicator 7. In this condition, the spatula 1′ is fully straightened and the surgeon is aware that, by keeping the contact on the entire contact area 3′, the pressure applied to the parenchyma is exactly equal to 45 mmHg. Thus, the surgeon who wished to apply a pressure of constantly less than 45 mmHg to the parenchyma P, should only have to carry out the retraction of the tissues without fully straightening the spatula 1′.

In light of the above, the spatula 1 allows the surgeon to precisely calibrate the maximum pressure exerted on the retracted tissues, with clear advantages in terms of the good outcome of the procedure.

The section narrowing 8 is between the third visual indicator 6 and the fourth visual indicator 7; as long as the surgeon grips the spatula 1′ at a visual indicator 4, 5 or 6 and therefore upstream of the section narrowing 8, any twists inadvertently imparted by the surgeon to portion 2 of spatula 1′ are transmitted to the portion 3 of spatula 1′ in a limited manner, i.e. with a smaller amplitude, precisely because of the elastic deformation undergone by the section narrowing 8, which is deformed. The function of the section narrowing 8 is therefore to keep the end 3 of the spatula 1′ in full contact with the parenchyma P as much as possible, with the entire surface of the end 3, by limiting or fully preventing the resting of the edge of the spatula 1′ only, which would more likely cause tissue damages.

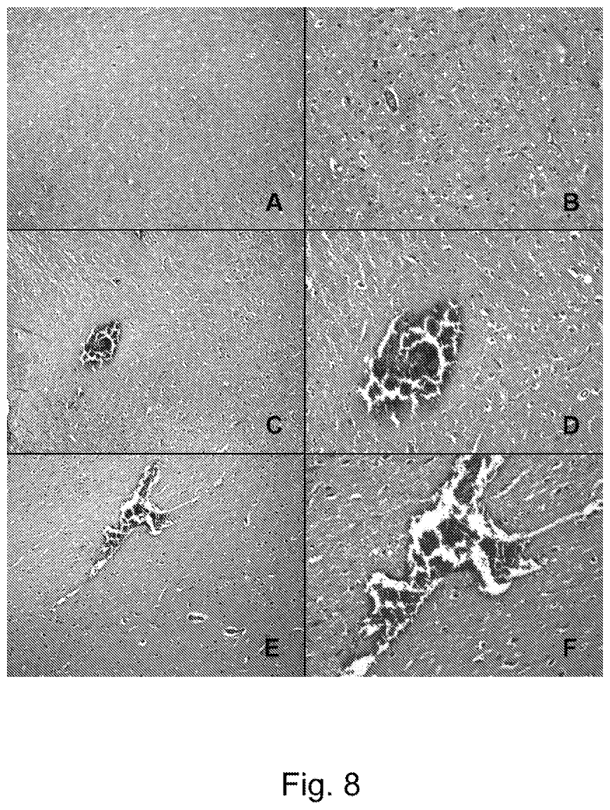

FIG. 8 shows microscopic images of samples of swine brain tissue, in particular parenchyma P. The samples were fixed in 10% neutral buffered formalin solution and incorporated in paraffin by using an automatic processor of tissues (Donatello Series 2, Diapath B G, Italy). 5 μm-thick sections were sectioned (Semi-automatic rotary microtome Galileo, Diapath, B G, Italy) and the sections were collected on poly-L-lysine-coated slides. The tissue sections were deparaffinized in xylene, rehydrated with gradually lower alcohol concentrations and stained with Haematoxylin-Eosin and Masson-Goldner by using an automatic stainer (Giotto, Diapath, B G, Italy). The slides were then observed under a 10× and 100× magnification microscope (Olympus) connected to a computer provided with image processing software.

In FIG. 8:
A is the image of a control section of the brain tissue, which has not undergone retraction, magnified by a 40× magnification factor;
B is the image of the same control section of the brain tissue as A, which has not undergone retraction, magnified by a 100× magnification factor;
C is the image of another section of the same brain tissue, a section that has undergone retraction with traditional spatula, magnified by a 40× magnification factor. A near-circular region of micro-bleeding due to the localized destruction of tissue is noticed. This lesion was caused by an excessive pressure applied locally with a conventional spatula;
D is the image of the same section of the brain tissue shown in B, but magnified by a 100× magnification factor. The localized lesion, where the micro-bleeding occurred, is clearly visible;
E is the image of another section of the brain tissue that has undergone retraction, magnified by a 40× magnification factor. A different region with lesion characterized by micro-bleeding due to the localized destruction of the tissue is noticed. This lesion was caused by an excessive pressure applied locally with a conventional spatula;
F is the image of the same section of the brain tissue shown in B, but magnified by a 100× magnification factor. The localized lesion, where the micro-bleeding occurred, is clearly visible and occupies a large part of the image.

The comparison of images C, D, E, F with images A and B gives an insight into the damages caused by an excessive pressure applied with a traditional spatula to tissues, particularly brain tissue.

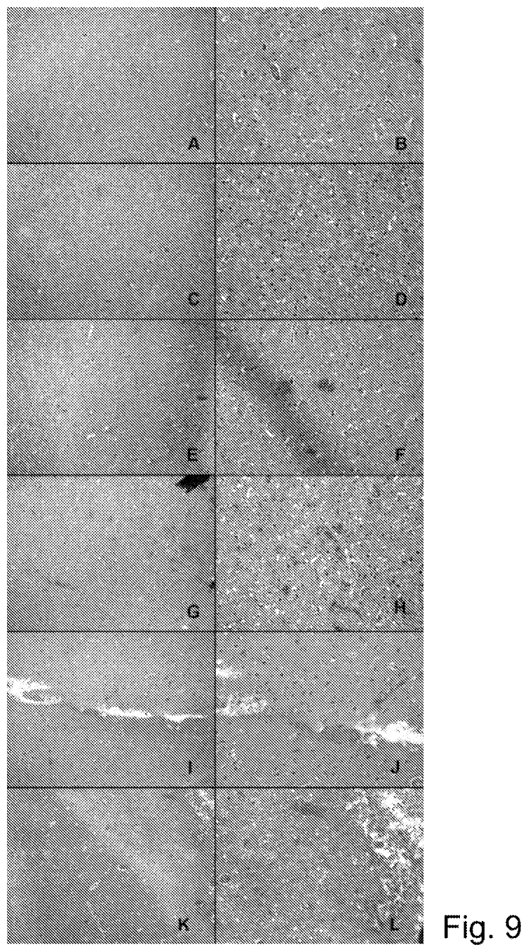

FIG. 9 shows the result of the retraction of the parenchyma P of a swine that can be achieved by using the spatula 1, 1′ according to the present invention. The samples were prepared as described above (Hematoxylin-Eosin staining).

In FIG. 9:
A is the image of a control section of parenchyma P, which has not undergone retraction, magnified by a 40× magnification factor;
B is the image of the same control section of the parenchyma P of A, which has not undergone retraction, magnified by a 100× magnification factor;
C is the image of another section of the same parenchyma P, a section that has undergone retraction, magnified by a 40× magnification factor. The retraction was carried out by using the spatula 1′ of the present invention, held by the surgeon at the first visual indicator 4, so as to apply a pressure on the parenchyma P always less than or at most equal (spatula in horizontal configuration) to 15 mmHg (1999.83 Pa), as described and shown in relation to FIG. 4. As can be seen, there are no lesions;
D is the image of the same section of the parenchyma P shown in C, but magnified by a 100× magnification factor;
E is the image of another section of the same parenchyma P, a section that has undergone retraction, magnified by a 40× magnification factor. The retraction was carried out by using the spatula 1′ of the present invention, held by the surgeon at the second visual indicator 5, so as to apply a pressure on the parenchyma P always less than or at most equal (spatula in horizontal configuration) to 22 mmHg (2933.084 Pa), as described and shown in relation to FIG. 5. As can be seen, there are micro-lesions, in particular bleeding in confined areas;
F is the image of the same section of the parenchyma P shown in E, but magnified by a 100× magnification factor;
G is the image of another section of the same parenchyma P, a section that has undergone retraction, magnified by a 40× magnification factor. The retraction was carried out by using the spatula 1′ of the present invention, held by the surgeon at the third visual indicator 6, so as to apply a pressure on the parenchyma P always less than or at most equal (spatula in horizontal configuration) to 30 mmHg (3999.66 Pa), as described and shown in relation to FIG. 6. As can be seen, there is more micro-bleeding and initial tissue destruction.
H is the image of the same section of the parenchyma P shown in G, but magnified by a 100× magnification factor;
I is the image of another section of the same parenchyma P, a section that has undergone retraction, magnified by a 40× magnification factor. The retraction was carried out by using the spatula 1′ of the present invention, held by the surgeon at the fourth visual indicator 7, so as to apply a pressure on the parenchyma P always less than 45 mmHg (5999.49 Pa), as described and shown in relation to FIG. 7. As can be seen, there are lesions, in particular bleeding and tissue destruction, in larger areas than the previous ones of images C-H;
J is the image of the same section of the parenchyma P shown in I, but magnified by a 100× magnification factor;
K is the image of another section of the same parenchyma P, a section that has undergone retraction, magnified by a 40× magnification factor. The retraction was carried out by using the spatula 1′ of the present invention. As can be seen, there are lesions, in particular bleeding, in the upper right hand, in confined areas;
L is the image of the same section of the parenchyma P shown in K, but magnified by a 100× magnification factor, in which the lesions are better visible.

From the comparison between FIG. 8 and FIG. 9, it is possible to see that the spatula 1′ has allowed to minimize the lesions to the parenchyma P under all conditions of use of the spatula 1′, offering de facto the same advantages as a sensor-equipped spatula but with the simplicity of a fully manual solution. Moreover, it has been emphasized that damages to brain parenchyma already begin at pressure values of 30 mmHg (3999.66 Pa), in accordance with the data currently present in the literature.

Additionally, the elastic hinge consisting of the section narrowing 8 helps to limit the twists transmitted to the tissue, so that the spatula 1, 1′ works flat and not cut with the edges; this expedient helps to achieve the excellent performance described above.

FIG. 10 is a schematic, not-to-scale, longitudinal sectional view of the straightened spatula 1 equipped with a light source 11, such as a LED. The LED 11 is arranged at the first end 2 and emits light beams 12 which are transmitted in the material of the spatula 1, e.g. polycarbonate, like in an optical fiber, until they come out at the second end 3, to illuminate the retracted tissues and help the surgeon to identify any micro-bleeding in, a timely manner.

1. A single-body manual surgical spatula which extends along a longitudinal axis between a first end and a second end and in which the thickness of the surgical spatula is less than the length and width of the surgical spatula,
wherein the surgical spatula can be held with one hand between the first end and the second end, and
wherein the surgical spatula is normally arched and in use is susceptible to bending, which tend to flatten or straighten it, in response to a pressure exerted at one end,
the surgical spatula being equipped with one or more tactile and/or visual indicators aligned along the longitudinal axis, each indicating a point of grip uniquely corresponding to a value known a priori of the bending strength of the surgical spatula.

2. The surgical spatula according to claim 1, wherein there are four tactile and/or visual indicators.

3. The surgical spatula according to claim 2, wherein the tactile and/or visual indicators correspond to maximum pressure values exerted by patient's tissues on a contact area of an end of the spatula, when the spatula is fully straightened during use, which are equal to 15 mmHg, 22 mmHg, 30 mmHg and 45 mmHg, respectively, equivalent to about 1999 Pa, 2933 Pa, 3999 Pa and 5999 Pa.

4. The surgical spatula according to claim 2, wherein an angular pitch between the tactile and/or visual indicators, which is calculated with the surgical spatula at rest, arched and with reference to the vertex of the angle at the center intercepted by the surgical spatula, corresponds to 4°-6°.

5. The surgical spatula according to claim 4, wherein a first tactile and/or visual indicator intercepts an angle at the center equal to 22° with the first end of the surgical spatula, and the fourth tactile and/or visual indicator intercepts an angle at the center equal to 36° with the first end of the surgical spatula.

6. The surgical spatula according to claim 1, wherein the tactile and/or visual indicators are bosses protruding from the top surface of the surgical spatula.

7. The surgical spatula according to claim 1, wherein the surgical spatula has a length equal to 15-20 cm, a width equal to 1-2 cm and a thickness equal to 1-4 mm, and wherein the radius of curvature of the spatula at rest is in the range of 150-200 mm.

8. The surgical spatula according to claim 1, wherein the spatula which is made with of a material having stiffness between 2 and 4 GPa.

9. The surgical spatula according to claim 1, wherein the spatula which is made of a transparent polymer material.

10. The surgical spatula according to claim 1, wherein the spatula which is made of a material selected from polycarbonate, polymethyl methacrylate, polystyrene, or low density polyethylene.

11. The surgical spatula according to claim 1, further comprising a section narrowing.

12. The surgical spatula according to claim 1, further comprising two notches, or indentations, opposite the longitudinal axis, each indentation being made in an edge of the surgical spatula, and in which the two notches define a section narrowing at which the surgical spatula twists.

13. The surgical spatula according to claim 12, wherein the two notches extend orthogonally to the longitudinal axis of the surgical spatula, one towards each other, at a portion of the surgical spatula intermediate between two tactile and/or visual indicators.

14. The surgical spatula according to claim 13, wherein the portion of surgical spatula intermediate between the two notches defines an elastic hinge that allows the relative rotation of the portion of spatula that is on one side of the notches with respect to the portion of spatula opposite the notches.

15. The surgical spatula according to claim 12, wherein said section narrowing extends along the longitudinal axis, and wherein said section narrowing extends in length for 2-5 mm and is at a position between 40% and 50% of the length of the surgical spatula, starting from the second end.

16. The surgical spatula according to claim 1, wherein the spatula is made of a transparent polymer material and equipped with a light source at the first end and wherein light produced by the light source is reflected within the surgical spatula, through the transparent material, and at emerges from the second end to illuminate the retracted tissues.

17. The surgical spatula according to claim 1, wherein the tactile and/or visual indicators constitute a graded scale of the bending strength of the surgical spatula corresponding to the strength that the surgical spatula provides to its straightening.

18. The surgical spatula according to claim 1, wherein the value of the strength that the surgical spatula provides to its straightening is certified for each tactile and/or visual indicator.
